(19) United States
(12) Patent Application Publication
(10) Pub. No.: US 2003/0216358 A1
(43) Pub. Date: Nov. 20, 2003

(54) Method for enhancing bone mineral density gain

(76) Inventors: Douglas Boyer Muchmore, LaJolla, CA (US); Yili Lu, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/332,179

(22) PCT Filed: Jul. 5, 2001
(86) PCT No.: PCT/US01/16515

(51) Int. Cl. A61K 31/66; A61K 31/675; A61K 31/445

(52) U.S. Cl. 514/89; 514/102; 514/319

(57) ABSTRACT

This invention relates to a method for enhancing bone mineral density gain acquired through previous bisphosphonate therapy comprising administering to a human in need thereof a bone-enhancing amount of raloxifene or a pharmaceutically acceptable salt or solvate thereof.

### BACKGROUND OF THE INVENTION

Current major diseases or conditions of bone which are of public concern include post-menopausal osteoporosis, senile osteoporosis, patients undergoing long-term treatment with corticosteroids, patients suffering from Cushings's syndrome, gonadal dysgenesis, periarticular erosions in rheumatoid arthritis, osteoarthritis, hypercalcemia of malignancy, osteopenia due to bone metastases, and periodontal disease. All of these conditions are characterized by bone loss, resulting from an imbalance between the degradation of bone (bone resorption) and the formation of new healthy bone. This turnover of bone continues normally throughout life and is the mechanism by which bone repairs and remodels. However, the conditions stated above may tip the balance towards bone loss such that the amount of bone resorbed is inadequately replaced with new bone, resulting in net bone loss.

One of the most common bone disorders is post-menopausal osteoporosis which affects an estimated 20 to 25 million women in the United States alone. Women after menopause experience an increase in the rate of bone turnover with resulting net loss of bone, as circulating estrogen levels decrease. The rate of bone turnover differs between bones and is highest in sites enriched with trabecular bone, such as the vertebrae and the proximal femur. The potential for bone loss at these sites immediately following menopause is up to 4-5% per year or more. The resulting decrease in bone mass and enlargement of bone spaces leads to increased fracture risk, as the mechanical integrity of bone deteriorates.

At present, there are 20 million people with detectable vertebral fractures due to osteoporosis and 250,000 hip fractures per year attributable to osteoporosis in the U.S. The latter case is associated with a 12% mortality rate within the first two years and 30% of the patients will require nursing home care after the fracture. Therefore, bone disorders are characterized by a noticeable mortality rate, a considerable decrease in the survivor's quality of life, and a significant financial burden to families and society.

Essentially all of the conditions listed above would benefit from treatment with agents which inhibit bone resorption. Bone resorption proceeds by the activity of specialized cells called osteoclasts. Osteoclasts are unique in their ability to resorb both the hydroxyapatite mineral and organic matrix of bone. They are somewhat similar to the cartilage resorbing cells, termed chondroclasts. It is for this reason that potent inhibitors of osteoclastic bone resorption may also inhibit the cell-mediated degradation of cartilage observed in rheumatoid arthritis and osteoarthritis.

Therapeutic treatments to impede net bone loss include the use of estrogens. Estrogens have been shown clearly to arrest the bone loss observed after menopause and limit the progression of osteoporosis; but patient compliance has been poor because of estrogen side-effects. These side effects include resumption of menses, mastodynia, increase in the risk of uterine cancer, and possibly an increase in the risk of breast cancer.

There are also a number of studies documenting the effects of the simultaneous combination of various antiresorptive agents. For example, the simultaneous administration of estrogen and the bisposponate alendronate has been shown to increase the bone mineral density (BMD) effects of estrogen alone in postmenopausal women with osteoporosis; Lindsay et al., J. Clin. Endocrinol. Metab. 84, 3076-3081 (1999). Similarly, a different bisphosphonate, etidronate, has shown additive BMD effects when administered concurrently with estrogens; Wimalawansa, Am. J. Med. 104, 219-226 (1998). Further, raloxifene HCl, a selective estrogen receptor modulator (SERM), has recently shown additive BMD effects during simultaneous treatment with alendronate; Johnell et al., J. Bone Miner. Res. 14 (Suppl.1), S157. However, the use of tamoxifen, another SERM, in premenopausal women, who have substantial endogenous estrogen levels, has resulted in reductions in BMD; Powles et al., J. Clin. Oncol. 14, 78-84 (1996). Therefore, the effect of two simultaneous antiresorptives is not readily predictable.

Furthermore, pharmaceutical compositions containing a bisphosphonate and an anti-resorptive agent for inhibiting bone loss are disclosed in European Patent Application Publ. No. 0 693 285 A2, inventors Black, L. J. and Cullinan, G. J., with a publication date of Jan. 24, 1996.

Third generation bisphosphonates such as alendronate are potent inhibitors of bone resorption, resulting in marked reductions in bone metabolic activity. Indeed, alendronate, especially when co-administered with estrogen, may result in suppression of bone turnover to levels that are below the mean for normal premenopausal women; Lindsay et al., J. Clin. Endocrinol. Metab. 84, 3076-3081 (1999). Prolonged profound reduction in bone turnover could theoretically result in an undesirable state of “frozen bone,” with reduction in bone formation and bone resorption to such an extent that normal repair and rejuvenation could become impaired. It is therefore desirable to find a bone loss treatment regimen that gives rapid clinical response without resulting in prolonged impairment of normal maintenance systems.

The current invention invokes the sequential use of a bisphosphonate followed by switching to raloxifene or pharmaceutically acceptable salt or solvate thereof, for the purpose of further increasing BMD in humans previously treated with a bisphosphonate. This benefit on BMD is associated with a shift of bone turnover markers back toward normal levels, reducing the risks of “frozen bone” that may be associated with excessive suppression of bone turnover by administration of highly potent antiresorptives either alone or in combination.

Clinical data supporting this invention have been collected. These data demonstrate that treatment with the bisphosphonate alendronate, for one year, followed sequentially by raloxifene alone, surprisingly results in further increases of BMD compared to alendronate followed by placebo. At the same time, bone turnover markers either return towards normal or remain suppressed but do not further decrease in the sequentially treated subjects. This surprising combination of effects, an increase BMD with return towards normal or maintenance without further suppression of bone turnover, makes this invention an ideal bone loss treatment regimen.

### SUMMARY OF THE INVENTION

This invention relates to a method for enhancing bone mineral density gain acquired through previous bisphosphonate therapy comprising administering to a human in need thereof a bone-enhancing amount of raloxifene or a pharmaceutically acceptable salt or solvate thereof.

Further, this invention relates to a method of inhibiting bone loss in a human in need thereof comprising administering a bone-enhancing amount of raloxifene or a pharmaceutically acceptable salt or solvate thereof subsequent to a course of bisphosphonate therapy.

This invention further relates to the use of raloxifene or a pharmaceutically acceptable salt or solvate thereof, in the preparation of a medicament for enhancing bone mineral density gain in a human, wherein said bone mineral density gain is acquired through previous bisphosphonate therapy.

In another embodiment, this invention relates to the use of raloxifene or a pharmaceutically acceptable salt or solvate thereof, in the preparation of a medicament for inhibiting bone loss in a human subsequent to a course of bisphosphonate therapy.

### DETAILED DESCRIPTION OF THE INVENTION

As used herein, the term “inhibit” is defined to include its generally accepted meaning which includes preventing, prohibiting, restraining, and slowing, stopping or reversing progression, or severity, and holding in check and/or treating existing characteristics. The present method includes both medical therapeutic and/or prophylactic treatment, as appropriate.

The phrase “enhance” is defined to include its generally accepted meaning which includes increasing, magnifying, amplifying, heightening, escalating, improving, boosting, intensifying and augmenting. Rises or gains in lumbar BMD with biphosphonate therapy for osteoporosis are generally in the range of four to eight percent compared to baseline with a majority of this rise occurring during the first twelve months of treatment. During the twelve months following discontinuation of therapy, the BMD is either generally stable or tending to decrease whereas markers of bone turnover return toward baseline. Increases in BMD in the order of 1-2% during the twelve months following discontinuation of bisphosphonate therapy, as is shown in the present invention, would be very much unexpected, especially if the bone turnover markers were either stable or returning toward baseline.

The phrase “bisphosphonate therapy” refers to administration of an effective amount of a bisphosphonate for a period of time sufficient to approach a plateau for bone density effect. A preferred length of time for a course of bisphosphonate therapy is at least six months, including treatment for six months to three years, preferably for at least about one year, with discontinuation of the therapy when appropriate to avoid excessive bone turnover.

The term “BMD” refers to bone mineral density.

The term “salt” or “pharmaceutically acceptable salt” refers to salts of the compounds of the above classes that are substantially non-toxic to living organisms. Typical pharmaceutically acceptable salts include those salts prepared by reaction of a compound of the above class with a pharmaceutically acceptable mineral or organic acid, or a pharmaceutically acceptable alkali metal or organic base, depending on the types of'substituents present on the compound.

Examples of pharmaceutically acceptable mineral acids which may be used to prepare pharmaceutically acceptable salts include hydrochloric acid, phosphoric acid, sulfuric acid, hydrobromic acid, hydroiodic acid, phosphorous acid and the like. Examples of pharmaceutically acceptable organic acids which may be used to prepare pharmaceutically acceptable salts include aliphatic mono and dicarboxylic acids, oxalic acid, carbonic acid, citric acid, succinic acid, phenyl-substituted alkanoic acids, aliphatic and aromatic sulfonic acids and the like. Such pharmaceutically acceptable salts prepared from mineral or organic acids thus include hydrochloride, hydrobromide, nitrate, sulfate, pyrosulfate, bisulfate, sulfite, bisulfite, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Many compounds of the above classes which contain a carboxy, carbonyl, or hydroxy or sulfoxide group may be converted to a pharmaceutically acceptable salt by reaction with a pharmaceutically acceptable alkali metal or organic base. Examples of pharmaceutically acceptable organic bases which may be used to prepare pharmaceutically acceptable salts include ammonia, amines such as triethanolamine, triethylamine, ethylamine, and the like. Examples of pharmaceutically acceptable alkali metal bases included compounds of the general formula MOZ, where M represents an alkali metal atom, e.g. sodium, potassium, or lithium, and Z represents hydrogen or C₁-C₄ alkyl, wherein C₁-C₄ alkyl represents a straight or branched chain alkyl radical of one to four carbon atoms such as methyl, ethyl, propyl, isopropyl and the like.

It should be recognized that the particular anion or cation forming a part of any salt of this invention is not critical, so long as the salt, as a whole, is pharmacologically acceptable and as long as the anion or cationic moiety does not contribute undesired qualities.

In addition, some of the compounds disclosed as useful in the methods of the present invention may form solvates with water or common organic solvents. Such solvates are included within the scope of the present invention and solvates thereof.

The class of compounds known as bisphosphonates includes those compounds which contains a di-phosphonic acid moiety separated by a carbon link and include a variety of side-chains, usually containing a basic function. The compounds have the following general structure:

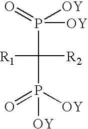

Y, R₁ and R₂ may be those substitutents as defined in U.S. Pat. No. 5,139,786, and EPO Publication 0416689A2, published Mar. 13, 1991, incorporated herein by reference, although not limited to such. A variety of bisphosphonic acids have been disclosed as being useful in the treatment and prevention of diseases involving bone resorption. Representative examples may be found in the following: U.S. Pat. Nos. 3,962,432, 4,054,598, 4,267,108, 4,327,039, 4,621,077, 4,624,947, 4,746,654, and 4,922,077, each of which is incorporated by reference herein as if fully set forth.

Pharmacologically, these compounds have been shown to slow or stop bone resorption by inhibiting osteoclast cell function. Several compounds of this class are currently undergoing clinical evaluation for the treatment of post-menopausal osteoporosis. Many of these compounds are also being evaluated for the treatment of Paget's Disease and hypercalcemia malignancy and several have been approved.

The art refers to three different generations of bisphosphonates. The first generation usually refers to the compound etidronate. This compound is being marketed for the treatment of Paget's disease and hypercalcemia malignacy.

The second generation of bisphosphonates refers to the compounds clodronate and pamidronate. Clodronate and pamidronate are both is marketed for Paget's disease and hypercalcemia maligancy. Pamidronate will probably be approved for osteoporosis in some European countries in the near future.

The third generation of bis-phosphonates refer to alendronate, residronate, and tiludronate and a host of lesser known compounds. Pharmacologically, these compounds are much more potent and are claimed to have fewer side-effects.

The structures of some bisphosphonate compounds are as follows:

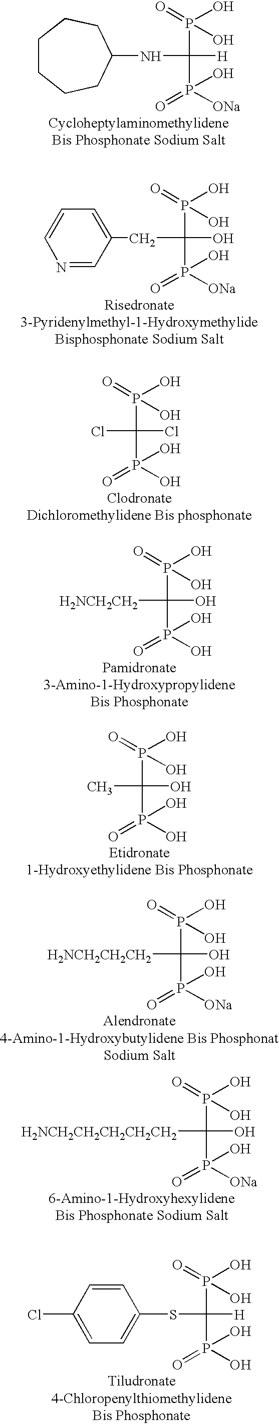

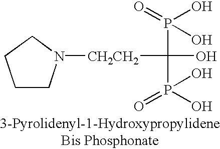

While some bisphosphonates are indicated for treating osteoporosis; they also appear to have potential detrimental side-effects. For example, bisphosphonates have the potential of inhibiting bone formation as well as resorption; they are poorly adsorbed via oral administration and are known to cause gastrointestinal irritation; they have extremely long half-lives in bone; they may all have the potential for causing osteomalacia; and there is concern as to the bio-mechanical strength of the bones treated with bisphosphonates.

During a course of bisphosphonate therapy, the amount of bisphosphonate administered to adult humans ranges from about 1 mg/day to about 400 mg/day. A course of bisphosphonate therapy generally lasts until a BMD plateau is achieved although such therapy may be used for repeated courses or continuously for an indefinite time. A preferred length of time for a course of bisphosphonate therapy is for at least about six months, including treatment for six months to three years, preferably for at least about one year, with discontinuation of the therapy when appropriate to avoid excessive suppression of bone turnover.

Preferred bisphosphonates are alendronate and risedronate, and salts thereof. Alendronate sodium is sold commercially as FOSAMAX®. Alendronate and salts thereof may be prepared according to known procedures such as those detailed in U.S. Pat. Nos. 4,621,077, 5,358,941, 5,681,590, 5,804,570, 5,849,726, and 6,008,207, each of which is incorporated by reference herein as if fully set forth. Risedronate sodium is sold commercially as ACTONEL®. Risedronate and salts thereof may be prepared according to known procedures such as those detailed in U.S. Pat. No. 5,583,122, herein incorporated by reference as if fully set forth.

The particular dosage of a bisphosphonate required to approach BMD plateau according to this invention will depend upon the particular circumstances of the conditions to be treated. Generally, an effective minimum daily dose for alendronate sodium is about 1, 2.5, 5, 10 or 20 mg. Typically, an effective maximum daily dose is about 200, 100, 80, 60 or 40 mg. A preferred daily dosage range is from about 5 mg to about 10 mg per day. For treatment of osteoporosis in postmenopausal women, the most preferred dosage is 10 mg once a day. For prevention of osteoporosis in postmenopausal women, the most preferred dosage is 5 mg once a day. For the treatment of glucocorticoid-induced osteoporosis in men and women, the most preferred dosage is 5 mg once a day, except for postmenopausal women not receiving estrogen, for whom the most preferred dosage is 10 mg once per day. For the treatment of Paget's disease of bone in men and women, the most preferred treatment regimen is 40 mg once a day for six months.

For risedronate, the preferred daily dose is about 1, 2.5, 5, 10, 15, or 30 mg. Typically, an effective maximum daily dose is about 300, 150, 120, 90, or 60 mg. For treatment of osteoporosis in postmenopausal women, the most preferred dosage ranges from about 2.5 to 20 mg per day, with 5 mg once per day being preferred. For the treatment of Paget's disease of bone in men and women, the most preferred treatment regimen is 30 mg once a day.

However, it will be understood that the amount of the bisphosphonate actually administered will be determined by a physician in light of the relevant circumstances including the condition to be treated, the choice of compounds to be administered, the age, weight, and response of the individual patient, the severity of the patient's symptoms and the chosen route of administration.

Raloxifene hydrochloride, which is 6-hydroxy-2-(4-hydroxyphenyl)-3-[4-(2-piperidinoethoxy)benzoyl]-benzo[b]thiophene, is sold commercially as EVISTA® and is represented by the formula:

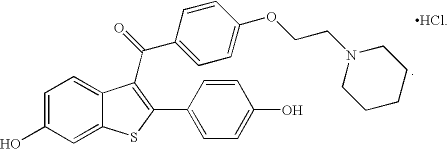

Raloxifene and salts and solvates thereof may be prepared according to known procedures, such as those detailed in U.S. Pat. Nos. 4,133,814, 4,418,068, 5,631,369, 5,731,327, 5,731,342, 5,750,688 and 5,977,383, each of which is incorporated by reference herein as if fully set forth. Preferred crystalline forms, particle sizes and pharmaceutical formulations are disclosed in U.S. Pat. Nos. 5,641,790, 5,731,327, 5,747,510, and 5,811,120, each of which is incorporated by reference herein as if fully set forth.

Pharmaceutical formulations can be prepared by procedures known in the art, such as, for example, in European Published Application 670162A1, published Sep. 6, 1995, and in WO 97/35571 published Oct. 2, 1997, both of which are herein incorporated by reference. For example, a compound of formula I can be formulated with common excipients, diluents, or carriers, and formed into tablets, capsules, and the like.

Examples of excipients, diluents, and carriers that are suitable for formulation include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonire; and lubricants such as talc, calcium and magnesium stearate and solid polyethyl glycols. Final pharmaceutical forms may be: pills, tablets, powders, lozenges, syrups, aerosols, saches, cachets, elixirs, suspensions, emulsions, ointments, suppositories, sterile injectable solutions, or sterile packaged powders, depending on the type of excipient used.

Additionally, raloxifene and its pharmaceutically acceptable salts are suited to formulation as sustained release dosage forms. The formulations can also be so constituted that they release the active ingredient only or preferably in a particular part of the intestinal tract, possibly over a period of time. Such formulations would involve coatings, envelopes, or protective matrices which may be made from polymeric substances or waxes.

The particular dosage of raloxifene or a pharmaceutically acceptable salt thereof required to constitute a “bone-enhancing amount” according to this invention will depend upon the particular circumstances of the conditions to be treated. Considerations such as dosage, route of administration, and frequency of dosing are best decided by the attending physician. Generally, an effective minimum dose for oral or parenteral administration of raloxifene or a pharmaceutically acceptable salt thereof is about 1, 5, 10, 15, or 20 mg. Typically, an effective maximum dose is about 800, 120, 60, 50, or 40 mg. A particularly effective amount is 60 mg of raloxifene hydrochloride (56 mg of free base) per day via an oral route of administration. Such dosages will be administered to a patient in need of treatment from one to three times each day or as often as needed to effectively enhance bone mineral density gain acquired through previous bisphoshonate therapy. Raloxifene hydrochloride may be administered for extended periods of time including six months to two years, specifically including about one year. Raloxifene hydrochloride may be used for repeated courses or continuously for an indefinite time.

The formulations which follow are given for purposes of illustration and are not intended to be limiting in any way. The total active ingredient in such formulations comprises from 0.1% to 99.9% by weight of the formulation. The term, “active ingredient” means a compound of formula I, or a pharmaceutical salt or solvate thereof, (preferably raloxifene hydrochloride). An even more preferred formulation of a compound of formula I would be raloxifene hydrochloride in the particular crystalline form, particle size, and composition illustrated in U.S. Pat. No. 5,731,327 and PCT application WO 97/35571 (Oct. 2, 1997) the teachings of each are incorporated by reference.

Formulation 1

Gelatin Capsules

| Ingredient | Quantity (mg/capsule) |
|---|---|
| Active Ingredient | 50-600 |
| Starch NF | 0-500 |
| Starch flowable powder | 0-500 |
| Silicone fluid 350 centistrokes | 0-15 |

The ingredients are blended, passed through a No. 45 mesh U.S. sieve, and filled into hard gelatin capsules.

Formulation 2

Tablets

| Ingredient | Quantity (mg/tablet) |
|---|---|
| Active Ingredient | 50-600 |
| Starch | 10-50 |
| Cellulose, microcrystalline | 10-20 |
| Polyvinylpyrrolidone | 5 |
| (as 10% solution in water) | |
| Sodium carboxymethyl cellulose | 5 |
| Magnesium stearate | 1 |
| Talc | 1-5 |

The active ingredient, starch, and cellulose are passed through a No. 45 mesh U.S. sieve and mixed thoroughly The solution of polyvinylpyrrolidone is mixed with the resultant powders which are then passed through a No. 14 mesh U.S. sieve. The granules thus produced are dried at 50-60° C. and passed through a No. 18 mesh U.S. sieve. The sodium carboxymethyl cellulose, magnesium stearate, and talc, previously passed through a No. 60 mesh U.S. sieve, are added to the above granules and thoroughly mixed. The resultant material is compressed in a tablet forming machine to yield the tablets.

Formulation 3

Aerosol

| Ingredient | Weight % |
|---|---|
| Active Ingredient | 0.50 |
| Ethanol | 29.50 |
| Propellant 22 | 70.00 |
| (Chlorodifluoromethane) | |

The active ingredient is mixed with ethanol and the mixture added to a portion of the propellant 22, cooled to −30° C. and transferred to a filling device. The required amount is then fed to a stainless steel container and diluted with the remainder of the propellant. The valve units are then fitted to the container.

Formulation 4

Suspension

| | |
|---|---|
| Ingredient | Weight/Volume |

| | | |
|---|---|---|
| Active Ingredient | 100 | mg |
| Sodium carboxymethyl | 50 | mg |
| cellulose | | |
| Syrup | 1.25 | mL |
| Benzoic acid solution (0.1 M) | 0.10 | mL |
| Flavor | q.v. | |
| Color | q.v. | |
| Purified water to total | 5 | mL |

Suspensions each containing 100 mg of a compound of formula I per 5 mL dose are prepared as follows: the active ingredient is passed through a No. 45 mesh U.S. sieve and mixed with the sodium carboxymethyl cellulose and syrup to form a smooth paste. The benzoic acid solution, flavor, and color diluted in water are added and mixture stirred thoroughly. Additional water is added to bring the entire mixture to the required volume.

### EXAMPLE

A phase 3, multicenter, double-blind, placebo-controlled, randomized clinical trial was conducted. Objectives of the trial included comparing the effects of 12 months treatment with raloxifene HCl (60 mg/day), alendronate (10 mg/day), or the combination of the two agents together versus placebo on BMD at the lumbar spine and at the hip and also metabolic markers of bone turnover. Additional objectives included comparing the effects of raloxifene 60 mg/day versus placebo during a second year of study on the offset of action of raloxifene or alendronate, as assessed by these same efficacy parameters.

Subjects were eligible for the trial if they were ambulatory postmenopausal women up to 75 years of age, inclusive, and had femoral neck BMD more than 2.0 standard deviations below the mean peak bone mass for healthy premenopausal women (i.e. T-score<−2.0). Subjects were ineligible if they had less than five years life expectancy, had current bone disorders (other than primary osteoporosis), had known or suspected history of carcinoma of the breast or other estrogen-dependent neoplasm, had history of cancer within five years of study, had abnormal uterine bleeding or endometrial thickness>5 mm by ultrasonography, had history of deep venous thrombosis or pulmonary embolism, had significant liver, kidney, gastroesophageal or intestinal malaborptive disease or any endocrine condition (other than type 2 diabetes or hypothyroidism) requiring pharmacologic therapy, were currently consuming excess of alcohol or drugs of abuse or were using or had recently received hormones or other therapies for osteoporosis.

Three hundred thirty-one (331) subjects were randomly assigned to one of four initial treatment groups: placebo, raloxifene 60 mg/day, alendronate 10 mg/day or raloxifene plus alendronate together. After one year of therapy all subjects were re-randomized to receive either placebo or raloxifene 60 mg/day for an additional year of study. All subjects received approximately 500 mg of elemental calcium plus 400-600 I.U. of vitamin D/day throughout the entire study. Subjects were seen at three monthly intervals, at which time assessment of medication compliance and adverse events were recorded. In addition subjects had BMD and biochemical markers of bone turnover (including serum osteocalcin) repeated at six monthly intervals. Additional procedures included annual mammograms, physical examinations and periodic assessments of health-related quality of life as well as sensory and neuromuscular tests to assess risks for falls.

Two hundred sixty-six (266) subjects completed the first therapy phase of 12 months and entered the extension phase. Among them, two hundred fifty-six (256) subjects completed 24 months of study. Rates of discontinuation during both study periods (0-12 months and 12-24 months) were not statistically significantly different across the four initial treatment group assignments. Comparing the group of subjects who were assigned to alendronate during year one and then randomly assigned to either raloxifene or placebo in year two, results of lumbar spine BMD, total hip BMD, femoral neck BMD, Serum Type I collagen fragment/creatinine ratio, N-telopeptide/creatinine ratio, bone specific alkaline phosphatase, and serum osteocalcin are given in the following tables:

TABLE 1
Lumbar Spine BMD (Mean percentage change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | 3.43% | 4.98% | 4.34% | 4.53% |
| | (n = 35) | (n = 35) | (n = 34) | (n = 32) |
| Aln/Rlx | 4.26% | 4.61% | 5.86% | 6.20% |
| | (n = 29) | (n = 30) | (n = 29) | (n = 27) |

¹Time from study start

TABLE 2
Lumbar Spine BMD (Mean percentage change from end
of 12 month alendronate treatment period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | −0.22% | 0.024 | −0.06% | 0.031 |
| | (n = 34) | | (n = 32) | |
| Aln/Rlx | 0.38%³ | | 1.48%³ | |
| | (n = 29) | | (n = 27) | |

¹Time from study start
²Between-group change tested by an ANOVA (analysis of variance) method
³Statistically significant within group change tested by Student's t-test (p < 0.05)

TABLE 3
Total Hip BMD (Mean percentage change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | 3.29% | 3.24% | 3.58% | 3.26% |
| | (n = 27) | (n = 27) | (n = 26) | (n = 24) |
| Aln/Rlx | 1.78% | 1.84% | 2.09% | 2.92% |
| | (n = 24) | (n = 25) | (n = 24) | (n = 23) |

¹Time from study start

TABLE 4
Total Hip BMD (Mean percentage change from end of
12 month alendronate treatment period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | 0.11% | 0.794 | −0.30% | 0.028 |
| | (n = 34) | | (n = 32) | |
| Aln/Rlx | 0.27% | | 1.08%³ | |
| | (n = 29) | | (n = 27) | |

¹Time from study start
²Between-group change tested by an ANOVA
³Statistically significant within group change tested by Student's t-test (p < 0.05)

TABLE 5
Femoral Neck BMD (Mean percentage change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | 2.71% | 3.70% | 3.79% | 3.51% |
| | (n = 35) | (n = 35) | (n = 34) | ( n = 31) |
| Aln/Rlx | 2.37% | 2.53% | 2.56% | 3.23% |
| | (n = 29) | (n = 30) | (n = 29) | (n = 28) |

¹Time from study start

TABLE 6
Femoral Neck BMD (Mean percentage change from end
of 12 month alendronate treatment period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | −0.01% | 0.826 | −0.38% | 0.202 |
| | (n = 34) | | (n = 31) | |
| Aln/Rlx | 0.23% | | 0.78% | |
| | (n = 29) | | (n = 28) | |

¹Time from study start
²Between-group change tested by an ANOVA

TABLE 7
Serum Type I Collagen Fragment/Creatinine ratio
(Median percentage change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | −73.2% | −71.5% | −44.0% | −14.0% |
| | (n = 26) | (n = 23) | (n = 32) | (n = 30) |
| Aln/Rlx | −74.2% | −82.2% | −57.4% | −53.4% |
| | (n = 22) | (n = 21) | (n = 24) | (n = 24) |

¹Time from study start

TABLE 8
Serum Type I Collagen Fragment/Creatinine ratio
(Median percentage change from end of 12 month alendronate
treatment period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | 161%³ | 0.458 | 349%³ | 0.243 |
| | (n = 22) | | (n = 19) | |
| Aln/Rlx | 113%³ | | 157%³ | |
| | (n = 21) | | (n = 20) | |

¹Time from study start
²Between-group change tested by an ANOVA on rank-transformed data
³Statistically significant within group change tested by Wilcoxon Signed Rank test (p < 0.05)

TABLE 9
N-teleopeptide/Creatinine ratio (Median percentage
change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | −62.4% | −60.1% | −30.4% | −9.1% |
| | (n = 34) | (n = 31) | (n = 32) | (n = 30) |
| Aln/Rlx | −58.9% | −63.1% | −25.5% | −38.5% |
| | (n = 29) | (n = 27) | (n = 27) | (n = 26) |

¹Time from study start

TABLE 10
N-telopeptide/Creatinine ratio (Median percentage
change from end of 12 month alendronate treatment period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | 90.3%³ | 0.398 | 131%³ | 0.156 |
| | (n = 30) | | (n = 28) | |
| Aln/Rlx | 56.7%³ | | 39.6%³ | |
| | (n = 26) | | (n = 25) | |

¹Time from study start
²Between-group change tested by an ANOVA on rank-transformed data
³Statistically significant within group change tested by Wilcoxon Signed Rank t-test (p < 0.05)

TABLE 11
Bone specific alkaline phosphatase (Median
percentage change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | −57.9% | −53.4% | −45.5% | −39.5% |
| | (n = 32) | (n = 31) | (n = 30) | (n = 28) |
| Aln/Rlx | −60.0% | −53.9% | −45.0% | −42.7% |
| | (n = 28) | (n = 27) | (n = 28) | (n = 27) |

¹Time from study start

TABLE 12
Bone specific alkaline phosphatase (Median
percentage change from end of 12 month alendronate treatment period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | 37.4%³ | 0.032 | 60.2%³ | 0.013 |
| | (n = 30) | | (n = 28) | |
| Aln/Rlx | 6.7%³ | | 7.1%³ | |
| | (n = 27) | | (n = 26) | |

¹Time from study start
²Between-group change tested by an ANOVA on rank-transformed data
³Statistically significant within group change tested by Wilcoxon Signed Rank t-test (p < 0.05)

TABLE 13
Serum Osteocalcin (Median percentage change from baseline)

| | | |
|---|---|---|
| | Alendronate Treatment | Placebo or Raloxifene |
| Treatment | Period¹ | Treatment Period¹ |

| Group | 6 months | 12 months | 18 months | 24 months |
|---|---|---|---|---|
| Aln/Pbo | −32.4% | −45.4% | −31.5% | −25.5% |
| | (n = 33) | (n = 31) | (n = 30) | (n = 28) |
| Aln/Rlx | −38.8% | −42.3% | −46.4% | −40.8% |
| | (n = 28) | (n = 27) | (n = 28) | (n = 27) |

¹Time from study start

TABLE 14
Serum Osteocalcin (Median percentage change from
end of 12 month alendronate treament period)

Placebo or Raloxifene Treatment Period¹

| | 18 months | | 24 months | |
|---|---|---|---|---|
| | | p-Value² | | p-Value² |
| | Mean % | (between- | Mean % | (between- |
| Treatment | Change | group) | Change | group) |
| Aln/Pbo | 38.2%³ | <0.001 | 52.1%³ | <0.001 |
| | (n = 30) | | (n = 28) | |
| Aln/Rlx | −2.75% | | 14.2%³ | |
| | (n = 27) | | (n = 26) | |

¹Time from study start
²Between-group change tested by an ANOVA on rank-transformed data
³Statistically significant within group change tested by Wilcoxon Signed Rank t-test (p < 0.05)

We claim:

1. A method for enhancing bone mineral density gain acquired through previous bisphosphonate therapy comprising administering to a human in need thereof a bone-enhancing amount of raloxifene or a pharmaceutically acceptable salt or solvate thereof.

2. A method according to claim 1 wherein said bisphosphonate is alendronate or a pharmaceutically acceptable salt thereof.

3. A method according to claim 1 wherein said bisphosphonate is alendronate sodium.

4. A method according to claim 1 wherein said bisphosphonate is risedronate or a pharmaceutically acceptable salt thereof.

5. A method according to claim 1 wherein said bisphosphonate is risedronate sodium.

6. A method according to claim 3 wherein said raloxifene is present as the hydrochloride salt thereof.

7. A method according to claim 6 wherein said raloxifene hydrochloride is administered in an amount of from about 10 mg to about 600 mg per day.

8. A method according to claim 7 wherein said raloxifene hydrochloride is administered in an amount of about 60 mg per day.

9. A method according to claim 1 wherein the term of said bisphosphonate therapy is at least about six months.

10. A method according to claim 1 wherein the term of said bisphosphonate therapy is from six months to three years.

11. A method according to claim 1 wherein the term of said bisphosphonate therapy is about one year.

12. A method according to claim 7 wherein the term of said bisphosphonate therapy is at least about six months.

13. A method according to claim 8 wherein the term of said bisphosphonate therapy is from six months to three years.

14. A method according to claim 8 wherein the term of bisphosphonate therapy is from six months to three years; and said alendronate sodium is administered in an amount of about 5 mg to about 10 mg per day.

15. A method according to claim 14 wherein the term of bisphosphonate therapy is about one year.

16. A method according to claim 14 wherein said raloxifene hydrochloride is administered for a term of at least about six months.

17. A method of inhibiting bone loss in a human in need thereof comprising administering a bone-enhancing amount of raloxifene or a pharmaceutically acceptable salt or solvate thereof subsequent to a course of bisphosphonate therapy.

18. A method according to claim 17 wherein said bisphosphonate is alendronate or a pharmaceutically acceptable salt thereof.

19. A method according to claim 18 wherein said bisphosphonate is alendronate sodium.

20. A method according to claim 17 wherein said bisphosphonate is risedronate or a pharmaceutically acceptable salt thereof.

21. A method according to claim 17 wherein said bisphosphonate is residronate sodium.

22. A method according to claim 19 wherein said raloxifene is present as the hydrochloride salt thereof.

23. A method according to claim 22 wherein said raloxifene hydrochloride is administered in an amount of from about 10 mg to about 600 mg per day.

24. A method according to claim 23 wherein said raloxifene hydrochloride is administered in an amount of about 60 mg per day.

25. A method according to claim 17 wherein the term of said bisphosphonate therapy is at least about six months.

26. A method according to claim 17 wherein the term of said bisphosphonate therapy is from six months to three years.

27. A method according to claim 17 wherein the term of said bisphosphonate therapy is about one year.

28. A method according to claim 23 wherein the term of said bisphosphonate therapy is at least about six months.

29. A method according to claim 24 wherein the term of said bisphosphonate therapy is from six months to three years.

30. A method according to claim 24 wherein the term of bisphosphonate therapy is from six months to three years; and said alendronate sodium is administered in an amount of about 5 mg to about 10 mg per day.

31. A method according to claim 30 wherein the term of bisphosphonate therapy is about one year.

32. A method according to claim 30 wherein said raloxifene hydrochloride is administered for a term of at least about six months.

33. The use of raloxifene or a pharmaceutically acceptable salt or solvate thereof, in the preparation of a medicament for enhancing bone mineral density gain in a human, wherein said bone mineral density gain is acquired through previous bisphosphonate therapy.

34. The use according to claim 33 wherein said bisphosphonate is risedronate, alendronate or a pharmaceutically acceptable salt thereof.

35. The use according to claims 33 or 34 wherein said bisphosphonate is alendronate sodium or risedronate sodium.

36. The use according to claims 33 to 35 wherein said raloxifene is present as the hydrochloride salt thereof.

37. The use according to claims 33 to 36 wherein said raloxifene hydrochloride is administered in an amount of from about 10 mg to about 600 mg per day.

38. The use according to claim 37 wherein said raloxifene hydrochloride is administered in an amount of about 60 mg per day.

39. The use according to claims 33 to 38 wherein the term of said bisphosphonate therapy is at least about six months.

40. The use according to claims 33 to 38 wherein the term of said bisphosphonate therapy is from six months to three years.

41. The use according to claim 33 wherein the term of said bisphosphonate therapy is about one year.

42. The use of raloxifene or a pharmaceutically acceptable salt or solvate thereof, in the preparation of a medicament for inhibiting bone loss in a human subsequent to a course of bisphosphonate therapy.

43. The use according to claim 42 wherein said bisphosphonate is risedronate, alendronate or a pharmaceutically acceptable salt thereof.

44. The use according to claim 42 wherein said bisphosphonate is alendronate sodium or risedronate sodium.

45. The use according to claims 42 to 44 wherein said raloxifene is present as the hydrochloride salt thereof.

46. The use according to claim 45 wherein said raloxifene hydrochloride is administered in an amount of from about 10 mg to about 600 mg per day.

47. The use according to claim 46 wherein said raloxifene hydrochloride is administered in an amount of about 60 mg per day.

48. The use according to claims 42 to 47 wherein the term of said bisphosphonate therapy is at least about six months.

49. The use according to claims 42 to 47 wherein the term of said bisphosphonate therapy is from six months to three years.

50. The use according to claims 42 to 47 wherein the term of said bisphosphonate therapy is about one year.

51. The use of raloxifene or a pharmaceutically acceptable salt or solvate thereof, in the preparation of a medicament for enhancing bone mineral density gain in a human previously treated with a bisphosphonate.

52. The use according to claim 51 wherein said bisphosphonate is risedronate, alendronate or a pharmaceutically acceptable salt thereof.

53. The use according to claims 51 or 52 wherein said bisphosphonate is alendronate sodium or risedronate sodium.

54. The use according to claims 51 to 53 wherein said raloxifene is present as the hydrochloride salt thereof.

55. The use according to claims 51 to 54 wherein said raloxifene hydrochloride is administered in an amount of from about 10 mg to about 600 mg per day.

56. The use according to claim 55 wherein said raloxifene hydrochloride is administered in an amount of about 60 mg per day.

57. The use according to claims 51 to 56 wherein the term of said bisphosphonate therapy is at least about six months.

58. The use according to claims 51 to 56 wherein the term of said bisphosphonate therapy is from six months to three years.

59. The use according to claim 51 wherein the term of said bisphosphonate therapy is about one year.

60. The use of raloxifene or a pharmaceutically acceptable salt or solvate thereof, in the preparation of a medicament for inhibiting bone loss in a human previously treated with a bisphosphonate.

61. The use according to claim 60 wherein said bisphosphonate is risedronate, alendronate or a pharmaceutically acceptable salt thereof.

62. The use according to claim 60 wherein said bisphosphonate is alendronate sodium or risedronate sodium.

63. The use according to claims 60 to 62 wherein said raloxifene is present as the hydrochloride salt thereof.

64. The use according to claim 63 wherein said raloxifene hydrochloride is administered in an amount of from about 10 mg to about 600 mg per day.

65. The use according to claim 64 wherein said raloxifene hydrochloride is administered in an amount of about 60 mg per day.

66. The use according to claims 60 to 65 wherein the term of said bisphosphonate therapy is at least about six months.

67. The use according to claims 60 to 65 wherein the term of said bisphosphonate therapy is from six months to three years.

68. The use according to claims 60 to 65 wherein the term of said bisphosphonate therapy is about one year.
